(19) United States
(12) Patent Application Publication
(10) Pub. No.: US 2010/0185137 A1
(43) Pub. Date: Jul. 22, 2010

(54) DEVICE FOR TREATMENT OF WATERING OF THE EYE

(75) Inventor: Eric Sarfati, Toulon (FR)

Correspondence Address: CANTOR COLBURN, LLP, 20 Church Street, 22nd Floor, Hartford, CT, 06103, US

(73) Assignees: Eric SARFATI, Toulon (FR); OPTISOINS SA, SION (CH)

(21) Appl. No.: 12/601,366

(22) PCT Filed: May 21, 2008
(86) PCT No.: PCT/FR08/00702

§ 371 Date: Mar. 29, 2010

(30) Foreign Application Priority Data
May 22, 2007 (FR) 0703626

(51) Int. Cl. A61M 1/00

(52) U.S. Cl. 604/8

(57) ABSTRACT

The present invention relates to a device (11) for treatment of watering of the eye, comprising a filiform element (13) and an intubation cannula (12), characterized in that at least one end of the filiform element (13) is provided with an elastic metal rod (14) comprising a curved distal end (15), said elastic metal rod being designed to slide inside said intubation cannula (12).

The present invention relates to a monocanalicular or bicanalicular probe that may be used in the treatment of watering of the eye.

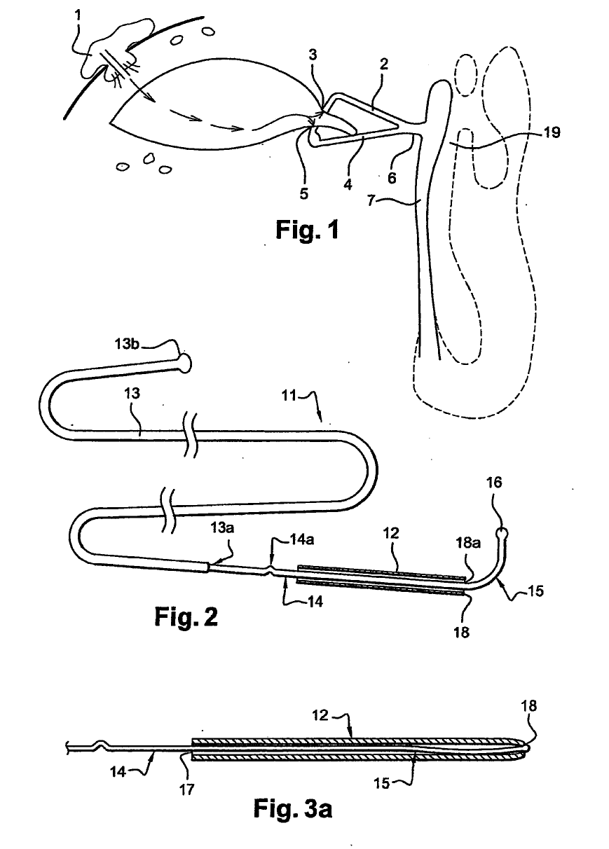

Watering of the eye can be due to overproduction of tears by the lacrimal gland. In this case, the intervention is on the gland to reduce its secretion. Another cause of watering of the eye is blockage, or stenosis, of the excretory lacrimal passages, namely the superior canaliculus, the inferior canaliculus, the common canaliculus and the nasolacrimal duct. The canaliculi correspond with the eye through meatuses located close to the nose in the upper and lower eyelids. Their function is to drain the tear fluid into the nose. FIG. 1 shows the lacrimal passages of the right eye of a patient. The figure shows the lacrimal gland 1, the superior canaliculus 2 and the superior meatus 3, the inferior canaliculus 4 and the inferior meatus 5, the common canaliculus 6, and the nasolacrimal duct 7. If it is possible to insert a probe past one of these ducts, these passages are catheterized and a probe is inserted, generally a filamentary part, such as a silicone tube, along which the tears can be drained into the nose, thus re-establishing the lacrimal flow.

One known technique of intubation employs a probe having a filamentary part, at each end of which is a straight metal stylet which acts as a guide for the filamentary part. In this method, the metal stylet on the first end is inserted for example into the inferior canaliculus via the inferior meatus, is steered into the nasolacrimal duct, and is then retrieved from the nasal fossa. In the second step, the metal stylet on the second end is inserted into the superior canaliculus via the superior meatus, steered into the nasolacrimal duct, and retrieved from the nasal fossa.

The problem with this method is that it is very difficult to retrieve each stylet from the nasal fossae. This task, repeated for each stylet, is often performed blind with pincers and is highly traumatizing for the flesh, which is injured and bleeds.

Another known intubation method employs a hollow cannula into which the filamentary part is inserted. The first step therefore is to insert the cannula into the inferior canaliculus via the inferior meatus and steer it into the nasolacrimal duct. The first end of the filamentary part is then slid into the cannula and retrieved from the nasal fossa. The same process is carried out with the second end and the superior canaliculus. However, this method also has the disadvantage of requiring the ends of the part to be retrieved blindly with pincers or a hook from the nasal fossa. This process is very bloody and traumatizing.

The present invention seeks to overcome these problems.

One object of the present invention is to provide a device, such as a monocanalicular or bicanalicular probe, that will enable the nontraumatic retrieval of the end or ends of said device from the nasal fossa or fossae.

The present invention relates to a device for treatment of watering of the eye, comprising a filamentary part and an intubation cannula, characterized in that at least one end of the filamentary part is provided with a spring metal rod having a curved distal end, said spring metal rod being designed to slide inside said intubation cannula.

Owing to the elastic nature of the metal rod of the device according to the invention, and its curved shape, the end of the metal rod automatically extends out of the cannula into the nasal fossa: to bring this about, the surgeon simply pushes on the filamentary part or on the proximal part of the rod near the proximal end of the cannula. The curved distal end of the metal rod of the device according to the invention is immediately visible to the surgeon, and the surgeon does not have to poke about for it blindly with pincers or a hook. The flesh, primarily the nasal tissues, are not injured and the operation causes much less trauma to the patient. Such an operation can thus be performed in the surgery.

In the present application, the “distal end” of a part is the end furthest from the user of the device, and “proximal end” is the end nearest the user of the device.

The filamentary part is preferably a silicone tube. Without implying any limitation, any flexible material capable of draining the tears could advantageously replace silicone for the filamentary part.

In one embodiment of the invention, the intubation cannula can be curved.

In a preferred embodiment of the invention, the spring metal rod comprises a straight portion and a curved portion, said curved portion comprising the curved distal end of the rod.

In a preferred embodiment of the invention, the distal end of the spring metal rod is curved into an arc of a circle.

In one particular embodiment of the invention, said curved distal end of the rod has a radius of curvature of 1 cm. More specifically, said curved distal end of the rod forms an arc of a circle describing from one quarter to three quarters of a circle.

In a preferred embodiment of the invention, the spring metal rod is made of a stainless steel, for instance a stainless steel having super-elastic properties such as the stainless steel sold under the trade name “304” by the Ugine company. What is meant by a metal or alloy having super-elastic properties is, for the purposes of the present application, any metal or alloy whose purely elastic deformability is from 5 to 10 times greater, or even 20 times greater, than that of steel.

In another embodiment of the invention, the spring metal rod is made of an alloy based on cobalt, chromium, nickel and molybdenum.

Because of the elasticity of the metal or alloy forming the metal rod of the device according to the invention, the rod can both be made to deform by the stress applied to it by the walls of the cannula, tending to align its curved distal end with the longitudinal axis of the cannula, when the rod is inside said cannula, and also resume its initial shape when this stress is removed, that is to say when it is extended from the cannula.

The filamentary part, which is preferably a silicone tube, is preferably from 20 to 50 cm long, preferably from 20 to 30 cm long.

The total length of the spring metal rod is advantageously approximately 18 cm. Alternatively, the total length of the rod is approximately 15 cm.

In one embodiment of the device according to the invention, said intubation cannula is fixed to said spring metal rod. Hence the curved distal end of the metal rod preferably has at its distal end an enlargement whose diameter is greater than the internal diameter of the distal orifice of the intubation cannula.

In one embodiment of the device according to the invention, said spring metal rod has in its proximal section a kink oriented in the same direction as the curvature of the curved distal end of said rod. This kink is a guide for the surgeon, indicating to him in which direction the curved distal end of the metal rod will emerge from the nasal fossa.

In one such embodiment of the device according to the invention, the sliding of said rod through the intubation cannula is limited by said enlargement on the curved distal end and by said kink. The spring metal rod is designed to slide inside said cannula between said enlargement and said kink only. The rod is thus fixed to the intubation cannula.

In one embodiment of the invention, each of the two ends of the filamentary part, preferably a silicone tube, is provided with a spring metal rod. In this case the device according to the invention is a bicanalicular probe.

In the case in which only one end of the filamentary part is provided with said spring metal rod, the device according to the invention is a monocanalicular probe.

Fitting a device according to the invention involves the following steps:

a) the intubation cannula containing the spring metal rod on one end of the filamentary part of the device according to the invention, is inserted through a meatus into a canaliculus and thence into the nasolacrimal duct. If using a spring metal rod with a kink, it is preferable to position the intubation cannula carefully by ensuring that the kink is uppermost;

b) the filamentary part or the proximal section of the metal rod is pushed in the distal direction, by acting on the proximal end of the cannula, until the curved distal end of the spring metal rod automatically extends out of the cannula in the nasal fossa. The curvature of the spring metal rod and the contact with the inferior wall of the nose cause the spring metal rod to extend out into the anterior nasal section. It is preferable, after performing step a) and before performing step b), to pull the cannula back a distance of about 1 cm;

c) the curved distal end of the spring metal rod is grasped, for example by the finger, in the nasal fossa and pulled to draw the filamentary part all the way into the lacrimal passageway. When performing this step, the surgeon can also grasp the cannula with, for instance, pincers or a hook.

If the device according to the invention is a bicanalicular probe, both ends of the filamentary part are provided with a spring metal rod. Both ends of the filamentary part of a bicanalicular probe are preferably fitted with an intubation cannula. In such a case, placing the device involves repeating steps a) to c) above via the second meatus into the second canaliculus with the second spring metal rod situated on the second end of the filamentary part of the device.

The invention thus makes it possible simply to push on the filamentary part or on the proximal section of the metal rod during intubation so that the curved distal end of the metal rod extends automatically, owing to its elasticity and the constrictions of the bony walls of the nose, in the correct direction into the nasal fossa and from there out of this fossa. Preferably, it is sufficient to push on the proximal section of the spring metal rod, for instance at a distance from the kink where relevant, and near the proximal section of the cannula.

Other features and advantages of the invention will be made clear by the following description of a particular embodiment, presented purely by way of non-restrictive example with reference to the drawings, in which:

FIG. 1 is a cross section through the lacrimal passages of the right eye of a patient

FIG. 2 is a cross section through a monocanalicular probe according to the invention

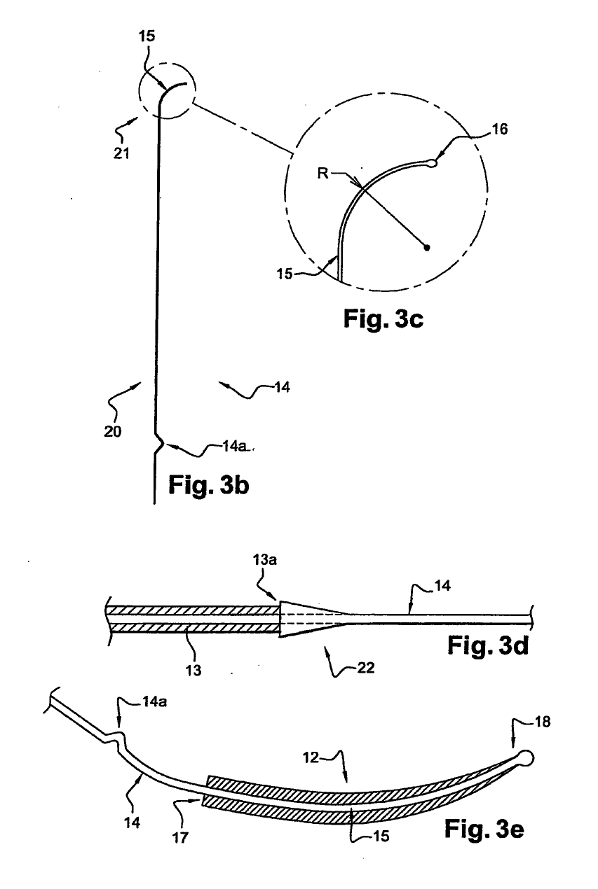

FIG. 3a is a partial cross section through the device according to the invention in which the curved distal end of the metal rod is stressed inside the intubation cannula

FIG. 3b is a side view of a particular embodiment of the spring metal rod of the device according to the invention

FIG. 3c is a view of a detail from FIG. 3b

FIG. 3d is a partial cross section through an embodiment of a device according to the invention in which the proximal end of the spring metal rod is crimped into the filamentary element by a crimping system

FIG. 3e is a partial cross section through an embodiment of the invention in which the intubation cannula is curved

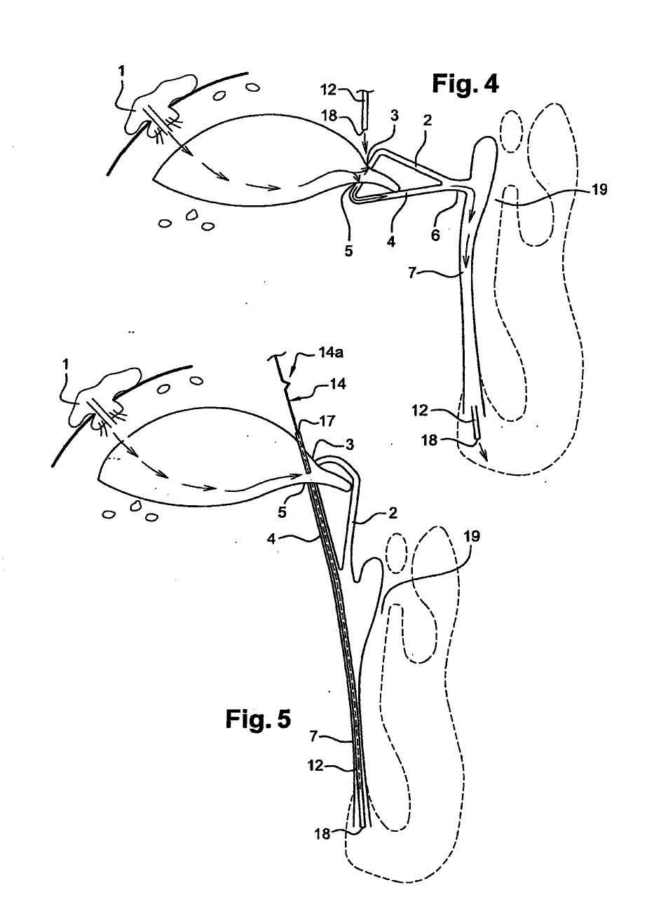

FIG. 4 illustrates the path of the intubation cannula from its insertion into a meatus to its emergence in the nasal fossa

FIG. 5 shows the intubation cannula in place with the curved distal end of the metal rod stressed inside the intubation cannula, and

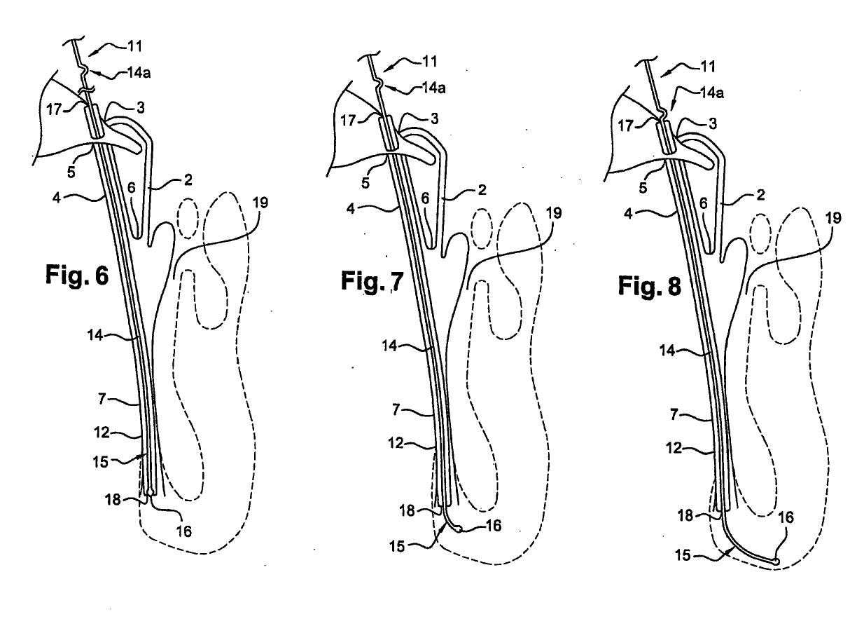

FIGS. 6 to 8 show the placement of the device according to the invention.

FIGS. 2 and 3a show a device 11 for treatment of watering of the eye, in the form of a monocanalicular probe. The device 11 illustrated in these figures comprises a filamentary part 13, preferably a silicone tube, and an intubation cannula 12.

One end 13a of the filamentary part 13 is provided with a spring metal rod 14 comprising a curved distal end 15. The other end 13b of the filamentary part has a meatic plug 13c of e.g. silicone or acrylic.

For instance, the spring metal rod 14 may be connected to the filamentary part 13, in particular to the silicone tube, by adhesive bonding or crimping.

FIG. 3d shows a crimping system 22 at the proximal end of the spring metal rod 14, the diameter of which varies regularly and progressively from the filamentary part 13 towards the spring metal rod 14, from a diameter approximately equal to or slightly greater than the diameter of the filamentary part 13 to a diameter equal to or slightly greater than the diameter of the spring metal rod 14. Besides the fact that it constitutes a means of crimping the spring metal rod 14 into the filamentary part 13, this kind of crimping system 22 makes it easier to pass the filamentary part into the lacrimal passages without injuring them.

The metal of the spring metal rod 14 is preferably a stainless steel, especially one having super-elastic properties such as the stainless steel sold under the trade name “304” by Ugine company. Other alternatives for the spring metal suitable for the rod of the device according to the invention are alloys based on cobalt, chromium, nickel, and molybdenum, such as the high elastic limit alloy sold under the trade name “PHYNOX” by IMPHY company, which has an elastic modulus of 210,000 MPa, or the alloy sold under the trade name “MP35N” by Carpenter company.

FIG. 3b shows an illustrative embodiment of the spring metal rod 14 with a straight portion 20 continued at its distal end by a curved portion 21 comprising the curved distal end 15 of the rod 14. The curved section 21 is an arc of a circle with a radius of curvature R of for example 1 cm. This curved section 21 ends in an enlargement 16 which has a diameter of between 0.04 cm and 0.06 cm. The diameter of the enlargement 16 is preferably greater than the internal diameter of the intubation cannula 12. The curved distal end 15 of the metal rod 14 has an enlargement 16 at its distal end with a diameter of for example 0.5 mm.

Thus, as FIG. 3a shows, the curved distal end 15 of the spring metal rod 14 deforms when fully inserted into the cannula 12 by the pressure of the walls of the cannula, which tend to align the curved distal end 15 of the rod with the longitudinal axis of the cannula 12. It then resumes its initial shape when extended out of the cannula 12 as shown in FIGS. 7 and 8.

The metal rod 14 has moreover in its proximal section a kink 14a oriented in the same direction as the curvature of the curved distal end 15 of said rod 14. This kink 14a can be replaced by any other device or mark that would show the orientation and direction of the curvature of the spring metal rod 14. As one example, given a spring metal rod 14 with a total length of 21 cm, the kink may for example be situated at a distance of approximately 16 cm from the distal end of the spring metal rod 14, and the intubation cannula may have a length of between 7 cm and 13 cm.

The total length of the spring metal rod 14 is advantageously approximately 18 cm. It is therefore very easy for the surgeon to retrieve the curved distal end 15 of the rod 14, using his fingers for example, when it emerges from the nasal fossa, and pull on it in order to engage the filamentary part 13 into a lacrimal passageway as shown in FIG. 8. Alternatively, the surgeon may use pincers to retrieve the curved distal end 15 of the rod 14 when it emerges from the nasal fossa.

To ensure that the spring metal rod 14 can slide properly, its diameter is less than the internal diameter of the intubation cannula. The diameter of the spring metal rod 14 is preferably approximately 0.25 mm.

The filamentary part 13, e.g. the silicone tube, is preferably from 20 to 50 cm long, preferably from 20 to 30 cm long. The filamentary part 13 may be a solid tube or a hollow tube.

The intubation cannula 12 may for example be approximately 7 cm long. It has a distal orifice 18a whose internal diameter is for example 0.5 mm. Thus, the diameter of the enlargement 16 on the curved distal end 15 of the spring metal rod 14 is greater than or equal to that of the distal orifice 18 of the cannula 12.

The metal rod 14 is thus fixed to the intubation cannula 12. The spring metal rod 14 can slide inside the intubation cannula between its enlargement 16 and its kink 14a. In the example illustrated, the filamentary part 13, the spring metal rod 14 and the intubation cannula 12 are inseparable, the enlargement 16 of the curved distal end 15, shaped for example like an egg, having a larger diameter than the internal diameter of the intubation cannula, and also the kink 14a not allowing the spring metal rod 14 to come all the way out.

FIG. 3e shows an embodiment of the invention in which the intubation cannula 12 is curved. The curvature of the intubation cannula is not necessarily the same as that of the spring metal rod 14.

FIG. 4 shows the path followed by the cannula 12 when intubated via the inferior canaliculus 6. The distal end 18 of the cannula 12 is inserted into the inferior meatus 5 and turned through 90° into the inferior canaliculus 4. When the distal end 18 of the cannula 12 bumps against the bone 19 of the nose, the cannula 12 is again turned through 90° to direct it down into the nasolacrimal duct 7 and into the nasal fossa. The cannula 12 is thus in the intubated position, as shown in FIG. 5.

When the cannula 12 is in this intubated position, it can be drawn back approximately 1 cm. The spring metal rod 14 is then slid as shown in FIG. 6. As can be seen in FIG. 5, the curved distal end 15 of the spring metal rod 14 deforms under the pressure of the walls of the cannula 12 and tends to align itself with the longitudinal axis of the cannula 12.

By pushing the proximal section of the rod 14 from the proximal end 17 of the cannula 12, the surgeon slowly advances the spring metal rod 14 towards the distal end 18 of the cannula 12 until the curved distal end 15 of this rod 14 emerges automatically forming with the longitudinal axis of the cannula 12 a curvature as shown in FIG. 7. This is due to the fact that as soon as the pressure of the walls of the cannula 12 is no longer acting on the distal end 15 of the spring metal rod 14, this distal end 15 returns to its initial curved shape and, being slid along the inferior walls of the nasal fossae, it emerges quite naturally into the nasal fossa.

In addition, since the kink 14a is oriented in the same direction as the curvature of the curved distal end 15 of the spring metal rod 14, the surgeon knows in which direction the curved distal end 15 will emerge into the nasal fossa.

The surgeon then continues to push on the proximal section of the rod 14 at the proximal end 17 of the cannula 12 and the curved distal end 15 of the spring metal rod 14 emerges completely from the nasal fossa as illustrated in FIG. 8. The surgeon can then easily retrieve the distal end 15 of this rod 14 by grasping it with the finger. A pincer could be used instead of the finger. The surgeon then pulls on the curved distal end 15 of this rod 14 to draw the filamentary part 13 all the way into this first lacrimal passageway.

If, in an embodiment not shown, the surgeon is placing a bicanalicular device, he withdraws the cannula 12 from the first end of the filamentary part 13 and the same operations as above are performed by introducing the cannula 12 from the second end of the filamentary part 13 via the superior meatus 3 through the superior canaliculus 2 and into the nasolacrimal duct. The curved distal end 15 of the metal rod of the second end of the filamentary part 13 is retrieved easily from the other nasal fossa as described earlier.

Once both cannulas and both metal rods have emerged into the nasal fossae, both ends of the filamentary part are cut off to remove the cannulas and spring metal rods and the two free ends of the filamentary part are tied together in a conventional way to hold the filamentary part in place.

The device according to the invention represents a clear improvement over existing devices by making possible the fitting of the filamentary part without causing trauma to the nasal flesh of the patient. The intervention is also made simpler from the point of view of the surgeon. In particular, with the device according to the invention a monocanalicular or bicanalicular probe can be fitted under ambulatory conditions such as the practitioner's surgery.

1. A device (11) for treatment of watering of the eye, comprising a filamentary part (13) and an intubation cannula (12), characterized in that at least one end of the filamentary part (13) is provided with a spring metal rod (14) having a curved distal end (15), said spring metal rod (14) being designed to slide inside said intubation cannula (12).

2. The device (11) as claimed in claim 1, characterized in that the distal end (15) of the spring metal rod (14) is curved into an arc of a circle.

3. The device (11) as claimed in claim 1 or 2, characterized in that the filamentary part (13) is a silicone tube.

4. The device (11) as claimed in any one of the preceding claims, characterized in that the spring metal rod (14) is made of a stainless steel.

5. The device (11) as claimed in any one of the preceding claims, characterized in that the spring metal rod (14) is made of an alloy based on cobalt, chromium, nickel and molybdenum.

6. The device (11) as claimed in any one of the preceding claims, characterized in that the filamentary part (13) is from 20 to 50 cm long, preferably from 20 to 30 cm long.

7. The device (11) as claimed in any one of the preceding claims, characterized in that the total length of the spring metal rod (14) is approximately 18 cm.

8. The device (11) as claimed in any one of the preceding claims, characterized in that said intubation cannula (12) is fixed to said spring metal rod (14).

9. The device (11) as claimed in the preceding claim, characterized in that the curved distal end (15) of the metal rod (14) has at its distal end an enlargement (16) whose diameter is greater than or equal to the internal diameter of the distal orifice (18a) of the intubation cannula (12).

10. The device (11) as claimed in any one of the preceding claims, characterized in that said spring metal rod (14) has in its proximal section a kink (14a) oriented in the same direction as the curvature of the curved distal end (15) of said rod (14).

11. The device (11) as claimed in claim 8 or 9, characterized in that the spring metal rod (14) is designed to slide inside said cannula (12) between said enlargement (16) and said kink (14a) only.

12. The device (11) as claimed in any one of the preceding claims, characterized in that each of the two ends (13a, 13b) of the filamentary part (13) is provided with a spring metal rod (14).
